(19) United States
(12) Patent Application Publication
(10) Pub. No.: US 2023/0404705 A1
(43) Pub. Date: Dec. 21, 2023

(54) GUIDED IMPLANT DRILL SYSTEM AND METHODS OF USE

(71) Applicant: Daniel S. Kim, Vancouver, WA (US)

(72) Inventor: Daniel S. Kim, Vancouver, WA (US)

(21) Appl. No.: 18/240,915

(22) Filed: Aug. 31, 2023

Related U.S. Application Data
(63) Continuation-in-part of application No. 17/377,133, filed on Jul. 15, 2021.
(60) Provisional application No. 63/402,823, filed on Aug. 31, 2022.
(60) Provisional application No. 63/052,067, filed on Jul. 15, 2020.

(51) Int. Cl. A61C 1/08; A61C 8/00

(52) U.S. Cl. CPC A61C 1/084; A61C 8/0089

(57) ABSTRACT

A guided implant drill system includes a guided implant drill bit with a cutting cylinder having counterclockwise helical cutting blades and flutes and a noncutting guide portion extending from the cutting cylinder, the guide portion having a diameter corresponding to a selected pilot drill bit. The guided implant drill bit may be constant diameter or tapered diameter. The system may include a set of guided implant drill bits having a range of cutting cylinder diameters and lengths. A method of use for a guided implant drill system is also provided.

### CROSS-REFERENCE TO RELATED APPLICATIONS

This application is a continuation-in-part of and claims priority of co-pending application Ser. No. 17/377,133, filed Jul. 15, 2021, which is a nonprovisional of, and claims priority to, provisional application 63/052,067 filed Jul. 15, 2020. This application is a nonprovisional of and claims priority to co-pending provisional application 63/402,823, filed Aug. 31, 2022. The disclosures of each of the preceding applications are hereby incorporated by reference into this application.

### FIELD OF THE INVENTION

The present invention relates to dental implant surgical bone graft devices and autogenous sinus lift surgery methods.

### BACKGROUND OF THE INVENTION

Conventional apparatus and methods for performing dental implant surgery are very basic, and vulnerable to errors created by the essentially free-hand drilling apparatus and methods widely used. The conventional procedure, after resecting the gum tissue, is to drill a pilot hole into the alveolar bone using a conventional bladed dental drill bit of small diameter, and then to drill successively wider holes using progressively larger conventional bladed drill bits until the required diameter to receive the intended implant anchor is achieved. There is nothing in their structure which would assist the surgeon in maintaining consistent alignment or hole diameter from step-to-step, especially if the surgeon attempted to switch immediately to using a large-diameter drill bit (e.g. in the 4 mm-8 mm range) after providing only a 2 mm or 2.2 mm pilot hole because the small-diameter pilot hole would provide little-to-no directional stability. The unguided free-hand nature of conventional dental drill bits requires multiple, incrementally increasing diameter, free-hand drilling steps.

Although the initial pilot hole provides some assistance in guiding the surgeon, each step essentially amounts to another free-handed drilling operation which introduces additional likelihood of errors in the angular and lateral displacement of the implant hole, with negative consequences for the patient—sometimes potentially severe consequences. Each drilling step introduces additional risk of causing unintended damage to the alveolar bone, through overheating and/or fracturing. Additionally, the requirement for multiple drilling steps substantially increases the time required to perform the procedure, thereby increasing the time the patient is subject to anesthesia—which increases surgical risk and patient discomfort—and reducing the number of patients the surgeon is able to assist in a given block of time, which increases costs.

In general, after teeth extracted the alveolar process is collapsed naturally during its healing procedures. The maxillary posterior teeth are located underneath the maxillary sinus cavity. When the maxillary posterior teeth are missing, and the alveolar process lost the support of the roots of the maxillary posterior teeth the sinus cavity is getting expanded by the force of air and enlarged into the area where the roots of the maxillary teeth used be. As a result, the wall of the maxillary sinus cavity where the maxillary posterior teeth used be is getting thinner.

When dentists plan to place dental implants on the maxillary posterior region to restore the missing teeth it is often found that there is not enough bone mass to embed the implant body. Therefore, bone graft is required into the lower part of maxillary sinus cavity to increase the vertical thickness of the alveolar bone where the maxillary posterior teeth used to be.

There are three materials used commonly for bone graft, autogenous bone graft from patient's own bone from the chin or iliac bone, allograft that is donated bone from others and xenograft from animal bone. Among those bone substitutes, autogenous bone graft is the most desirable material to get the best result. Therefore, years ago autogenous bone graft was performed for the sinus lift for patients that could afford this expensive procedure. But its extensive surgical procedures to get the bone from the patient's own body like the chin bone or iliac bone is very intrusive physically and mentally for the patient, prolongs healing time, and is very expensive such that many (or most) patients cannot afford the procedure. Because of the inconvenience, expense, intrusiveness and discomfort, and the additional surgical time required, allograft or xenograft has been more widely used in dental practices, which are bone harvested from deceased donors and bone harvested from certain animal species.

Thus, there is a need for an improved dental surgical drill system which reduces risk of misalignment errors, reduces the stress imposed on the alveolar bone during drilling procedures, and reduces the time required to perform such procedures, and provides at least a portion of bone graft material by allograft from the osteotomy site.

### SUMMARY AND ADVANTAGES

A guided implant drill system includes a guided implant drill bit with a cutting cylinder having counterclockwise helical cutting blades and flutes and a noncutting guide portion extending from the cutting cylinder, the guide portion having a diameter corresponding to a selected pilot drill bit. The guided implant drill bit may be constant diameter or tapered diameter. The system may include a set of guided implant drill bits having a range of cutting cylinder diameters and lengths. A method of use for a guided implant drill system is also provided.

A method for performing an osteotomy in preparation for installing a dental implant anchor using a set of a guided implant drill bit and guided implant final drill bit includes the steps of:

Step 1: the pilot drill bit is commonly used to initiate drilling on the alveolar bone and to guide for the final drill.

Step 2: set the pilot drill bit on the alveolar bone with the right direction and depth.

Step 3: as soon as the tip of the pilot drill bit drills through the bone, stop drilling and make sure there is no puncture on the maxillary sinus cavity membrane/Schneiderian membrane. If there is a puncture happened use an artificial membrane to block the puncture.

Step 4: the new sinus drill bit is equipped with the guiding tip and sinus stopper. Also the spiral of the blades are designed to cut the alveolar bone to make the bone chips and push them up instead push them out.

Step 5: the new sinus drill bit drills up along the guiding hole that the pilot drill made and push the bone chips up into the sinus cavity through the hole the pilot drill made under the maxillary sinus cavity membrane/Schneiderian membrane.

Step 6: the new sinus drill bit is reached up to the desired depth and the mixture of bone chips and saline solution occupied in the space created by the pressure of saline solution from the dental handpiece.

Step 7: the implant body is placed into the hole and in the sinus under the maxillary sinus cavity membrane/Schneiderian membrane.

The guided implant drill system described and claimed provides important advantages over existing apparatus and methods, including but not limited to: (1) providing directional support for a dental surgeon in carrying out the widening portion of an osteotomy in preparation for a dental implant while providing improved channels for irrigation to minimize risk of bone necrosis due to heat generation and provide efficient bone chip movement; (2) reducing the number of drilling cycles required to complete a preparatory osteotomy; and, (3) providing apparatus and means to complete a preparatory osteotomy with minimum risk of unintentional additional removal of bone from the upper portion of the osteotomy location. Additional benefits include: 1) autogenous bone graft material generated from the osteotomy sit itself, with lessened potential need for artificial bone material; 2) quicker healing times with lower risk of complications; 3) reduced costs to patients, including by eliminating expense for artificial bone graft material and shortened surgical procedures; 4) during the drilling portion of the osteotomy, bone graft material is automatically inserted through the hole made by the pilot drilling; and, 6) the smooth guide portion tip greatly reduces risk of perforating of the sinus membrane.

### BRIEF DESCRIPTION OF THE DRAWINGS

The accompanying drawings, which are incorporated into and constitute a part of this specification, illustrate one or more embodiments of the invention and, together with the detailed description, serve to explain the principles and implementations of the invention.

FIG. 1 is a side view of a first embodiment of a dental implant drill bit system.

FIG. 1a is an end view of a first embodiment, from the guide portion terminal end.

FIG. 1b is a cutaway end view of a first embodiment, from the cutting cylinder portion first end.

FIG. 1c is an end view of a first embodiment, showing the guide portion terminal end flutes.

FIG. 1d is a closeup side view of a first embodiment, showing the stopper portion and cutting cylinder portion base.

FIG. 2 is a side view of a second embodiment of a dental implant drill bit system.

FIG. 2a is an end view of a second embodiment, from the guide portion terminal end.

FIG. 2b is a cutaway end view of a second embodiment, from the cutting cylinder portion first end.

FIG. 2c is an end view of a second embodiment, showing the guide portion terminal end flutes.

FIG. 2d is a closeup side view of a second embodiment, showing the stopper portion and cutting cylinder portion base.

FIG. 3 shows multiple views of a third embodiment of a guided implant drill bit, with a straight cutting cylinder portion.

FIG. 4 shows multiple views of a fourth embodiment of a guided implant drill bit, with a straight cutting cylinder portion.

FIG. 5 shows multiple views of a fifth embodiment of a guided implant drill bit, with a straight cutting cylinder portion.

FIG. 6 shows multiple views of a sixth embodiment of a guided implant drill bit, with a straight cutting cylinder portion.

FIG. 7 shows multiple views of a seventh embodiment of a guided implant drill bit, with a straight cutting cylinder portion.

FIG. 8 shows multiple views of an eighth embodiment of a guided implant drill bit, with a straight cutting cylinder portion.

FIG. 9 shows multiple views of a ninth embodiment of a guided implant drill bit, with a tapered cutting cylinder portion.

FIG. 10 shows multiple views of a tenth embodiment of a guided implant drill bit, with a tapered cutting cylinder portion.

FIG. 11 shows multiple views of an eleventh embodiment of a guided implant drill bit, with a tapered cutting cylinder portion.

FIG. 12 shows multiple views of a twelfth embodiment of a guided implant drill bit, with a tapered cutting cylinder portion.

FIG. 13 shows multiple views of a thirteenth embodiment of a guided implant drill bit, with a tapered cutting cylinder portion.

FIG. 14 shows multiple views of a fourteenth embodiment of a guided implant drill bit, with a tapered cutting cylinder portion.

FIG. 15 shows a side view of an embodiment of a pilot drill bit.

FIG. 16 shows a side view of an embodiment of a pilot drill bit.

FIG. 17 shows a simplified cutaway view of an osteotomy procedure, with the pilot drill bit (dental driver not shown) selected and prepared to initiate drilling.

FIG. 18 shows a simplified cutaway view of an osteotomy procedure, with the pilot drill bit (dental driver not shown) at maximum penetration.

FIG. 19 shows a simplified cutaway view of an osteotomy procedure, with the dental implant drill bit (dental driver not shown) selected and prepared to initiate drilling.

FIG. 20 shows a simplified cutaway view of an osteotomy procedure, with the dental implant drill bit (dental driver not shown) initiating drilling.

FIG. 21 shows a simplified cutaway view of an osteotomy procedure, with the dental implant drill bit (dental driver not shown) at maximum penetration.

FIG. 22 shows a simplified cutaway view of an osteotomy procedure, with the dental implant mounted into the osteotomy location.

### REFERENCE NUMBERS

The following table provides a summary of the Reference Numbers used in the Detailed Description and the Drawings:

| Ref. Nr. | Description |
|---|---|
| 100 | First Embodiment |
| 102 | Shank Portion |
| 104 | Cutting Cylinder Portion |
| 106 | Guide Portion |
| 108 | Rotational Axis |
| 110 | Shank Portion First End |
| 112 | Shank Portion Second End |
| 114 | Cutting Cylinder Portion First End |
| 116 | Cutting Cylinder Portion Second End |
| 118a | Cutting Cylinder Portion Helical Blade |
| 118b | Cutting Cylinder Portion Helical Blade |
| 118c | Cutting Cylinder Portion Helical Blade |
| 120 | Stopper Portion |
| 122a | Cutting Cylinder Portion Helical Blade First End |
| 122b | Cutting Cylinder Portion Helical Blade First End |
| 122c | Cutting Cylinder Portion Helical Blade First End |
| 124a | Cutting Cylinder Portion Helical Blade Second End |
| 124b | Cutting Cylinder Portion Helical Blade Second End |
| 124c | Cutting Cylinder Portion Helical Blade Second End |
| 126 | Stopper Portion First Beveled Surface |
| 128 | Stopper Portion Second Beveled Surface |
| 130a | Cutting Cylinder Portion Helical Flute |
| 130b | Cutting Cylinder Portion Helical Flute |
| 130c | Cutting Cylinder Portion Helical Flute |
| 132a | Cutting Cylinder Portion Helical Flute First End |
| 132b | Cutting Cylinder Portion Helical Flute First End |
| 132c | Cutting Cylinder Portion Helical Flute First End |
| 134a | Cutting Cylinder Portion Helical Flute Second End |
| 134b | Cutting Cylinder Portion Helical Flute Second End |
| 134c | Cutting Cylinder Portion Helical Flute Second End |
| 146 | Guide Portion First End |
| 148 | Guide Portion Terminal End |
| 156 | Guide Portion Ogival Base |
| 158 | Guide Portion Diameter |
| 160 | Cutting Cylinder Portion Diameter |
| 164 | Guide Portion Outer Surface |
| 166 | Guide Portion Length |
| 168 | Cutting Cylinder Length |
| 170a | Guide Portion Noncutting Helical Blade |
| 170b | Guide Portion Noncutting Helical Blade |
| 170c | Guide Portion Noncutting Helical Blade |
| 180a | Guide Portion Helical Flute |
| 180b | Guide Portion Helical Flute |
| 180c | Guide Portion Helical Flute |
| 200 | Second Embodiment |
| 202 | Shank Portion |
| 204 | Cutting Cylinder Portion |
| 206 | Guide Portion |
| 208 | Rotational Axis |
| 210 | Shank Portion First End |
| 212 | Shank Portion Second End |
| 214 | Cutting Cylinder Portion First End |
| 216 | Cutting Cylinder Portion Second End |
| 218a | Cutting Cylinder Portion Helical Blade |
| 218b | Cutting Cylinder Portion Helical Blade |
| 218c | Cutting Cylinder Portion Helical Blade |
| 220 | Stopper Portion |
| 222a | Cutting Cylinder Portion Helical Blade First End |
| 222b | Cutting Cylinder Portion Helical Blade First End |
| 222c | Cutting Cylinder Portion Helical Blade First End |
| 224a | Cutting Cylinder Portion Helical Blade Second End |
| 224b | Cutting Cylinder Portion Helical Blade Second End |
| 224c | Cutting Cylinder Portion Helical Blade Second End |
| 226 | Stopper Portion First Beveled Surface |
| 228 | Stopper Portion Second Beveled Surface |
| 230a | Cutting Cylinder Portion Helical Flute |
| 230b | Cutting Cylinder Portion Helical Flute |
| 230c | Cutting Cylinder Portion Helical Flute |
| 232a | Cutting Cylinder Portion Helical Flute First End |
| 232b | Cutting Cylinder Portion Helical Flute First End |
| 232c | Cutting Cylinder Portion Helical Flute First End |
| 234a | Cutting Cylinder Portion Helical Flute Second End |
| 234b | Cutting Cylinder Portion Helical Flute Second End |
| 234c | Cutting Cylinder Portion Helical Flute Second End |
| 246 | Guide Portion First End |
| 248 | Guide Portion Terminal End |
| 256 | Guide Portion Ogival Base |
| 258 | Guide Portion Diameter |
| 260 | Cutting Cylinder Portion Tapered Surface |
| 262 | Cutting Cylinder Portion Base Diameter |
| 264 | Guide Portion Outer Surface |
| 266 | Guide Portion Length |
| 268 | Cutting Cylinder Length |
| 270a | Guide Portion Noncutting Helical Blade |
| 270b | Guide Portion Noncutting Helical Blade |
| 270c | Guide Portion Noncutting Helical Blade |
| 280a | Guide Portion Helical Flute |
| 280b | Guide Portion Helical Flute |
| 280c | Guide Portion Helical Flute |
| 300 | Guided Implant Drill Bit Third Embodiment |
| | (Straight Cutting Cylinder Portion) |
| 400 | Guided Implant Drill Bit Third Embodiment |
| | (Straight Cutting Cylinder Portion) |
| 500 | Guided Implant Drill Bit Third Embodiment |
| | (Straight Cutting Cylinder Portion) |
| 600 | Guided Implant Drill Bit Third Embodiment |
| | (Straight Cutting Cylinder Portion) |
| 700 | Guided Implant Drill Bit Third Embodiment |
| | (Straight Cutting Cylinder Portion) |
| 800 | Guided Implant Drill Bit Third Embodiment |
| | (Straight Cutting Cylinder Portion) |
| 900 | Guided Implant Drill Bit Third Embodiment |
| | (Tapered Cutting Cylinder Portion) |
| 1000 | Guided Implant Drill Bit Third Embodiment |
| | (Tapered Cutting Cylinder Portion) |
| 1100 | Guided Implant Drill Bit Third Embodiment |
| | (Tapered Cutting Cylinder Portion) |
| 1200 | Guided Implant Drill Bit Third Embodiment |
| | (Tapered Cutting Cylinder Portion) |
| 1300 | Guided Implant Drill Bit Third Embodiment |
| | (Tapered Cutting Cylinder Portion) |
| 1400 | Guided Implant Drill Bit Third Embodiment |
| | (Tapered Cutting Cylinder Portion) |
| 1500 | Pilot Drill Bit |
| 1600 | Pilot Drill Bit |

### DETAILED DESCRIPTION

Before beginning a detailed description of the subject invention, mention of the following is in order. When appropriate, like reference materials and characters are used to designate identical, corresponding, or similar components in different figures. The figures associated with this disclosure typically are not drawn with dimensional accuracy to scale, i.e., such drawings have been drafted with a focus on clarity of viewing and understanding rather than dimensional accuracy.

In the interest of clarity, not all of the routine features of the implementations described herein are shown and described. It will, of course, be appreciated that in the development of any such actual implementation, numerous implementation-specific decisions must be made in order to achieve the developer's specific goals, such as compliance with application and business related constraints, and that these specific goals will vary from one implementation to another and from one developer to another. Moreover, it will be appreciated that such a development effort might be complex and time-consuming but would nevertheless be a routine undertaking of engineering for those of ordinary skill in the art having the benefit of this disclosure.

Use of directional terms such as “upper,” “lower,” “above,” “below”, “in front of,” “behind,” etc. are intended to describe the positions and/or orientations of various components of the invention relative to one another as shown in the various Figures and are not intended to impose limitations on any position and/or orientation of any embodiment of the invention relative to any reference point external to the reference.

Those skilled in the art will recognize that numerous modifications and changes may be made to the exemplary embodiment(s) without departing from the scope of the claimed invention. It will, of course, be understood that modifications of the invention, in its various aspects, will be apparent to those skilled in the art, some being apparent only after study, others being matters of routine mechanical, chemical and electronic design. No single feature, function or property of the exemplary embodiment(s) is essential. Other embodiments are possible, their specific designs depending upon the particular application. As such, the scope of the invention should not be limited by the particular embodiments herein described but should be defined only by the appended claims and equivalents thereof.

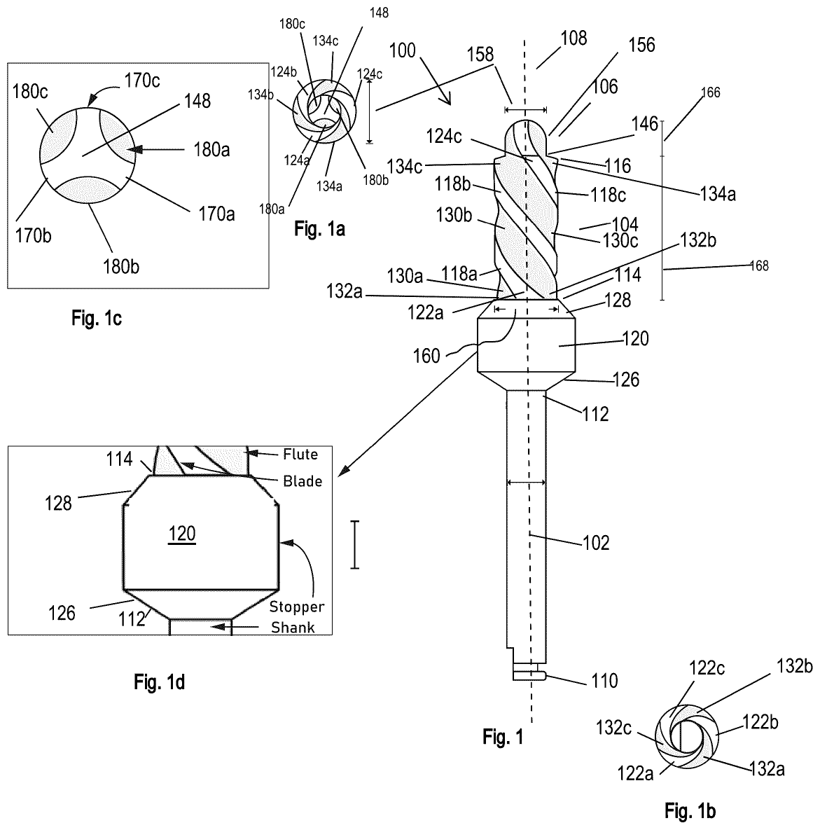

Referring to FIGS. 1-1d, a first embodiment of a guided implant drill system, with straight cutting cylinder, is shown. Guided implant drill bit 100 includes a shank portion 102, a stopper portion 120, a cutting cylinder portion 104 and a guide portion 106. Shank portion 102 is adapted to couple to a selected dental drill driver or class of dental drivers, the shank portion extending from a shank first end 110 to a shank second end 112. The centerlines of shank portion 102, cutting cylinder portion 104 and guide portion 106 align and together define the rotational axis 108 of the guided implant drill bit 100.

In the embodiment, stopper portion 120 comprises a stop collar of slightly wider diameter than cutting cylinder portion first end 114, and includes first and second beveled surfaces 126, 128, to minimize tissue damage when contacting gingival tissues and to provide improved visibility to the surgeon.

Cutting cylinder portion 104 extends along rotational axis 108 from cutting cylinder portion first end 114 rigidly connected to stopper portion 120 to cutting cylinder portion second end 116, defining a cutting cylinder length 168. Cutting cylinder portion 104 includes a plurality of helical blades 118a, 118b and 118c, each extending from a blade first end 122a, 122b, 122c, respectively, to a blade second end 124a, 124b, 124c, respectively. In the embodiment, blades 118a-c are disposed around cutting cylinder portion 104 at 1200 intervals, defining an equal plurality of coextensive helical flutes 130a, 130b and 130c, each flute 130a-c extending from a respective flute first end 132a, 132b, 132c to a respective flute second end 134a, 134b, 134c. In the embodiment, the blade first ends 122a-c are coextensive with cutting cylinder portion first end 114, and the blade second ends 124a-c are coextensive with cutting cylinder portion second end 116.

In the embodiment, the bases of blades 118a-c, corresponding to the depths of flutes 130a-c, are flush with the circumference of guiding tip 106. In the embodiment, blades 118a-c and corresponding flutes 130a-c each have a pitch of 360° per 8 mm around the cutting cylinder portion circumference from cutting cylinder first end 114 to cutting cylinder second end 116. Cutting cylinder blades 118a-c and flutes 130a-c have a reversed pitch—i.e. when rotating in the clockwise direction (viewed from the shank portion first end 110) the blades 118a-c are pushing cut material forward rather than back, as with a traditional drill bit orientation. Pushing cut material, consisting of bone chips, forward causes the bone chips to fill in to the gap between the sinus bone and the maxillary sinus cavity membrane/Schneiderian membrane, thereby providing additional material to bond the dental implant. Additionally, saline solution applied by the surgeon for lubrication and cooling is forced forward to ensure it moves through the cutting contact area to prevent heat damage to bone and other tissue in the vicinity.

The outer edges of blades 118a-c define a constant cutting cylinder portion diameter 160. The cutting cylinder portion diameter 160 is selected based upon the size of the selected dental implant anchor (or other surgical considerations). Depth markings may be provided along cutting cylinder 106 as visual guides for the surgeon.

Guide portion 106 extends from guide portion first end 146 proximate cutting cylinder portion second end 116 to a terminal end 148, defining guide portion length 166. Guide portion length 166 is selected to provide adequate engagement without leaving an excessive requirement of finish drilling. Guide portion 106 includes a plurality of noncutting helical blades 170a, 170b, 170c and corresponding helical flutes 180a, 180b, 180c which align with cutting cylinder blades 118a-c and flutes 130a-c, in order to maintains a fluid communication/vent pathway from the region ahead of guided drill bit 100. In the embodiment, guide portion noncutting flukes 170a-c join at the terminal end 148 of guide portion 106. Guide portion terminal end 148 approximates a spherically blunted ogival profile.

Guide portion 106 defines a cylinder of constant diameter 158 from guide portion first end 146 to ogival base 156. The outer surface 164 of guide portion 106 is generally smooth to avoid abrasion and excessive frictional heating, and guide portion blades 170a-c do not have cutting edges, as it is intended to provide alignment for the cutting portion through the pilot hole and maintain fluid communication, but not to cut bone material. Guide portion diameter 158 matches the cutting diameter of the selected pilot drill.

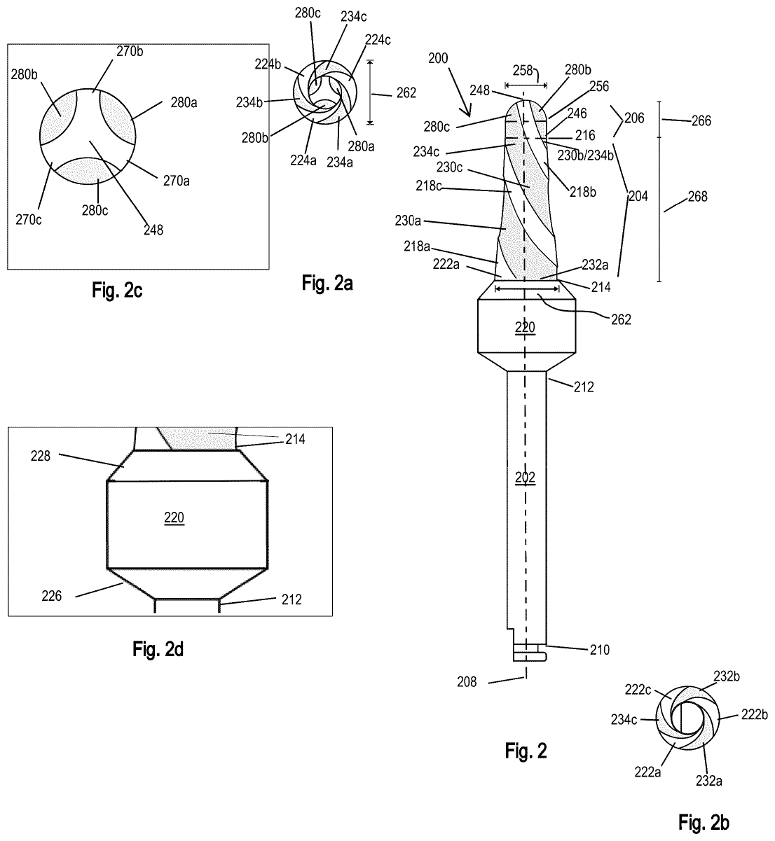

Referring to FIGS. 2-2d, a second embodiment of a guided implant drill system 200, with tapered cutting cylinder, is shown. Guided implant drill bit 200 includes a shank portion 202, a stopper portion 220, a cutting cylinder portion 204 and a guide portion 206. Shank portion 202 is adapted to couple to a selected dental drill driver or class of dental drivers, the shank portion extending from a shank first end 210 to a shank second end 212. The centerlines of shank portion 202, stopper portion 220, cutting cylinder portion 204 and guide portion 206 align and together define the rotational axis 208 of the guided implant drill bit 200.

In the embodiment, stopper portion 220 comprises a stop collar of slightly wider diameter than cutting cylinder portion first portion 214, and includes first and second beveled surfaces 226, 228, to minimize tissue damage when contacting gingival tissues and to provide improved visibility to the surgeon.

Cutting cylinder portion 204 extends along rotational axis 208 from cutting cylinder portion first end 214 rigidly connected to stopper portion 220 to cutting cylinder portion second end 216, defining a cutting cylinder length 268. Cutting cylinder portion 204 includes a plurality of helical cutting blades 218a, 218b and 218c, each extending from a blade first end 222a, 222b, 222c, respectively, to a blade second end 224a, 224b, 224c, respectively. In the embodiment, blades 218a-c are disposed around cutting cylinder portion 204 at 120° intervals, defining an equal plurality of coextensive helical flutes 230a, 230b and 230c, each flute 230a-c extending from a respective flute first end 232a, 232b, 232c to a respective flute second end 234a, 234b, 234c. In the embodiment, the blade first ends 222a-c are coextensive with cutting cylinder portion first end 214, and the blade second ends 224a-c are coextensive with cutting cylinder portion second end 216. The widths of helical cutting blades 218a-c and helical flutes 230a-c vary proportionally with the taper of cutting cylinder portion 204, being wider at their respective first ends 222a-c and 232a-c and progressively narrower toward their respective second ends 224a-c and 234a-c.

In the embodiment, blades 218a-c and corresponding flutes 230a-c each have a pitch of 360° per 8 mm around the cutting cylinder portion circumference from cutting cylinder first end 214 to cutting cylinder second end 216. Cutting cylinder blades 218a-c and flutes 230a-c have a reversed pitch—i.e. when rotating in the clockwise direction (viewed from the base of shank portion 210) the blades 218a-c are pushing cut material forward rather than back, as with a traditional drill bit orientation. Pushing cut material, consisting of bone chips, forward causes the bone chips to fill into the gap between the sinus bone and the maxillary sinus cavity membrane/Schneiderian membrane, thereby providing additional material to bond the dental implant. Additionally, saline solution applied by the surgeon for lubrication and cooling is forced forward to ensure it moves through the cutting contact area to prevent heat damage to bone and other tissue in the vicinity.

Cutting cylinder portion 204 tapers from cutting cylinder portion first end 214 defining a base diameter 262 to cutting cylinder portion second end 216, for use with a tapered dental anchor implant. The outer edges of blades 218a-c define a tapered cutting cylinder surface 260. Depth markings may be provided along cutting cylinder 206 as visual guides for the surgeon. The selected taper angle of tapered cutting cylinder portion 206 is determined by the selected dental implant.

Guide portion 206 extends from guide portion first end 246 proximate cutting cylinder portion second end 216 to a terminal end 248, defining guide portion length 266. Guide portion length 266 is selected to provide adequate engagement without leaving an excessive requirement of finish drilling. Guide portion 206 includes a plurality of blades 270a, 270b, 270c, and corresponding flutes 280a, 280b, 280c which align with cutting cylinder blades 218a-c and flutes 230a-c, in order to maintains a fluid communication/vent pathway from the region ahead of guided drill bit 200. In the embodiment, guide portion noncutting flukes 270a-c join at the terminal end 248 of guide portion 206. Guide portion terminal end 248 approximates a spherically blunted ogival profile.

Guide portion 206 defines a cylinder of constant diameter 258 from guide portion first end 246 to ogival base 256, with guide portion diameter 258 the same as the diameter of tapered cutting cylinder portion second end 216. The outer surface 264 of guide portion 206 is generally smooth to avoid abrasion and excessive frictional heating, and guide portion blades 270a-c do not have cutting edges, as it is intended to provide alignment for the cutting portion through the pilot hole and maintain fluid communication, but not to cut bone material. Guide portion diameter 258 matches the cutting diameter of the selected pilot drill.

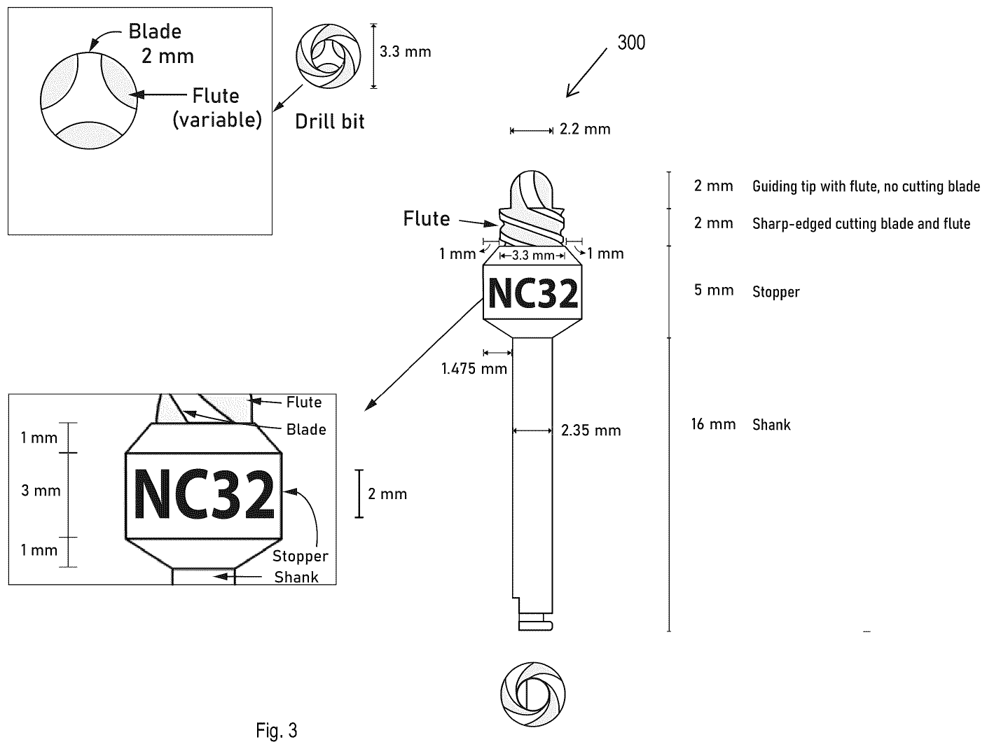

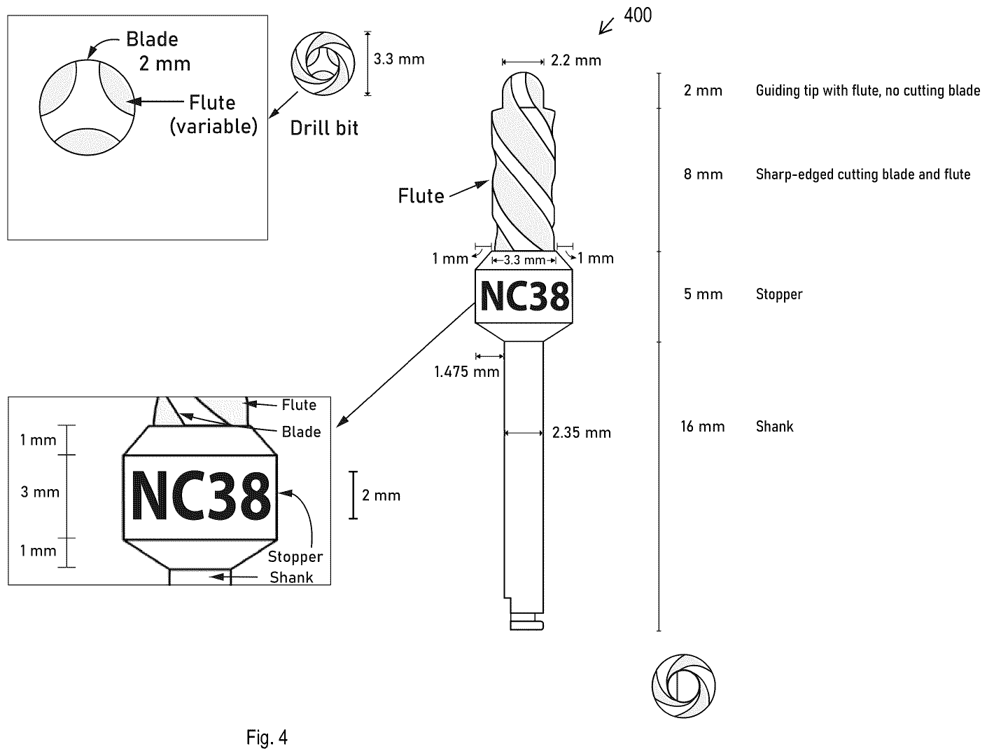

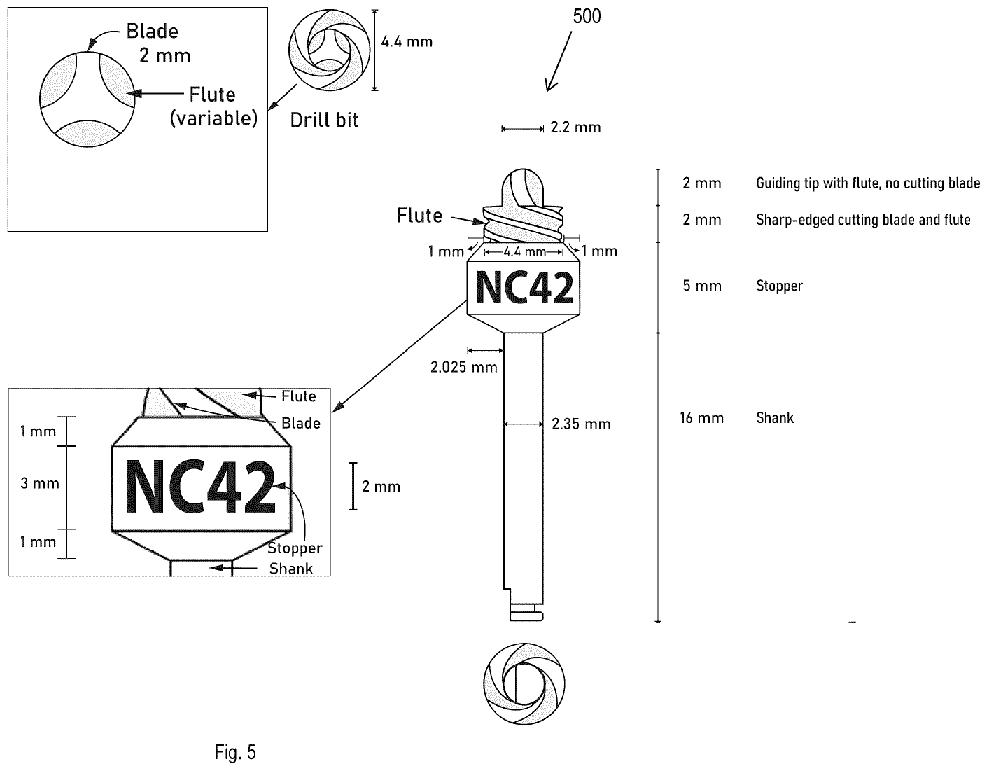

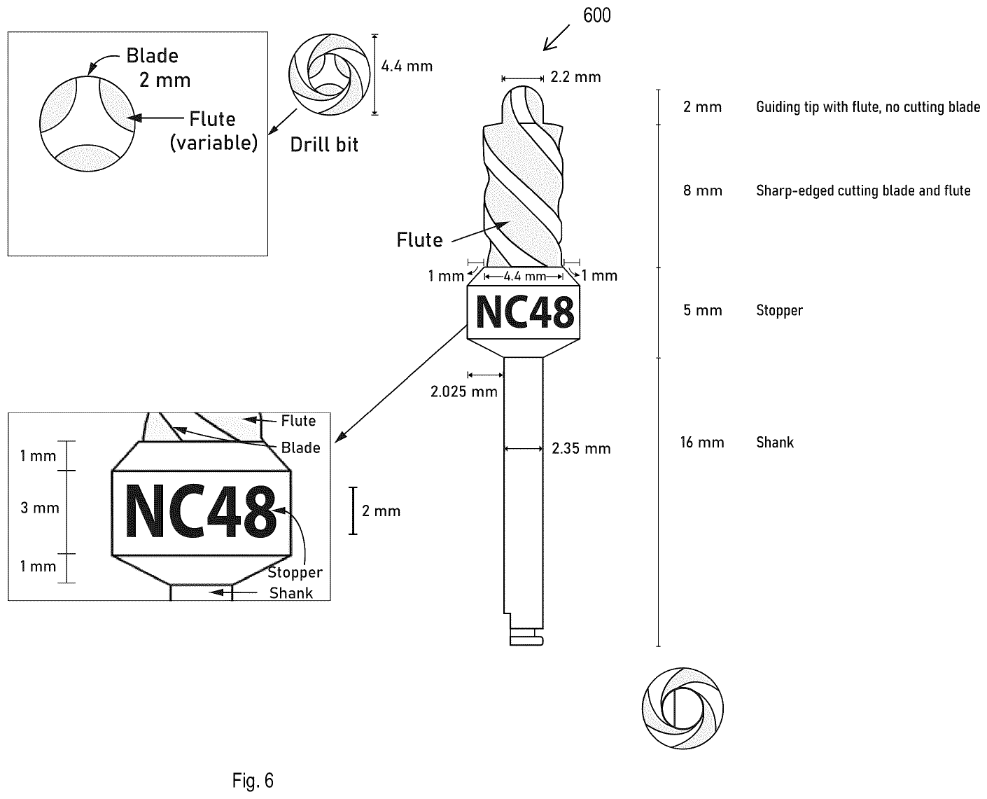

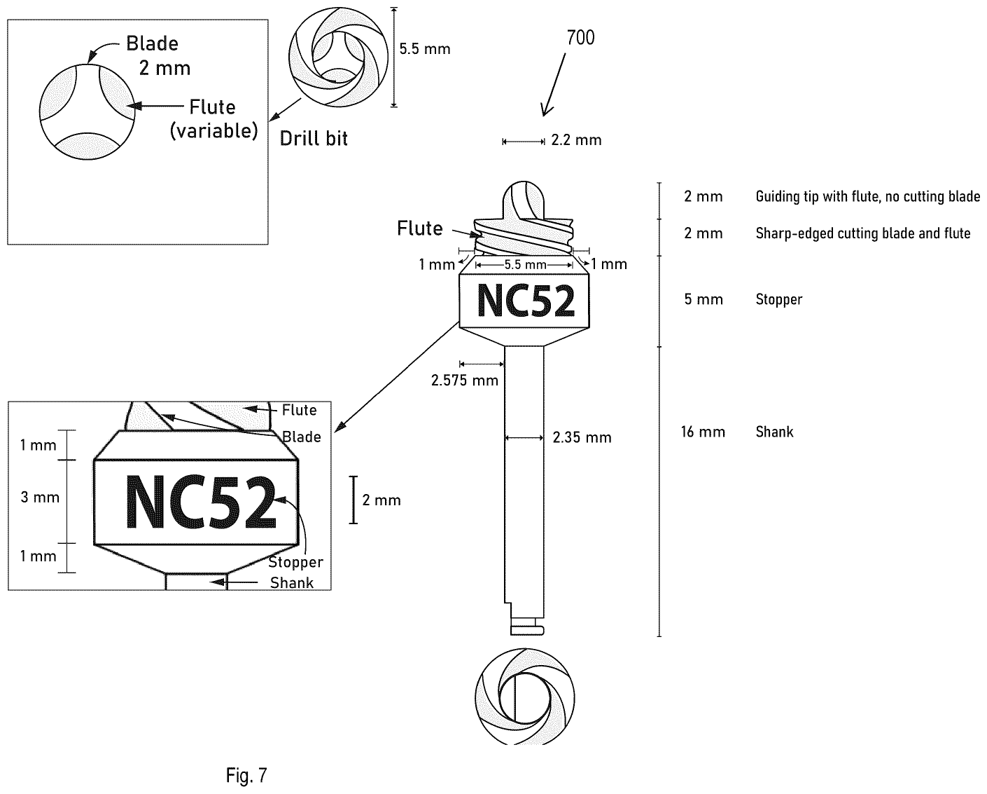

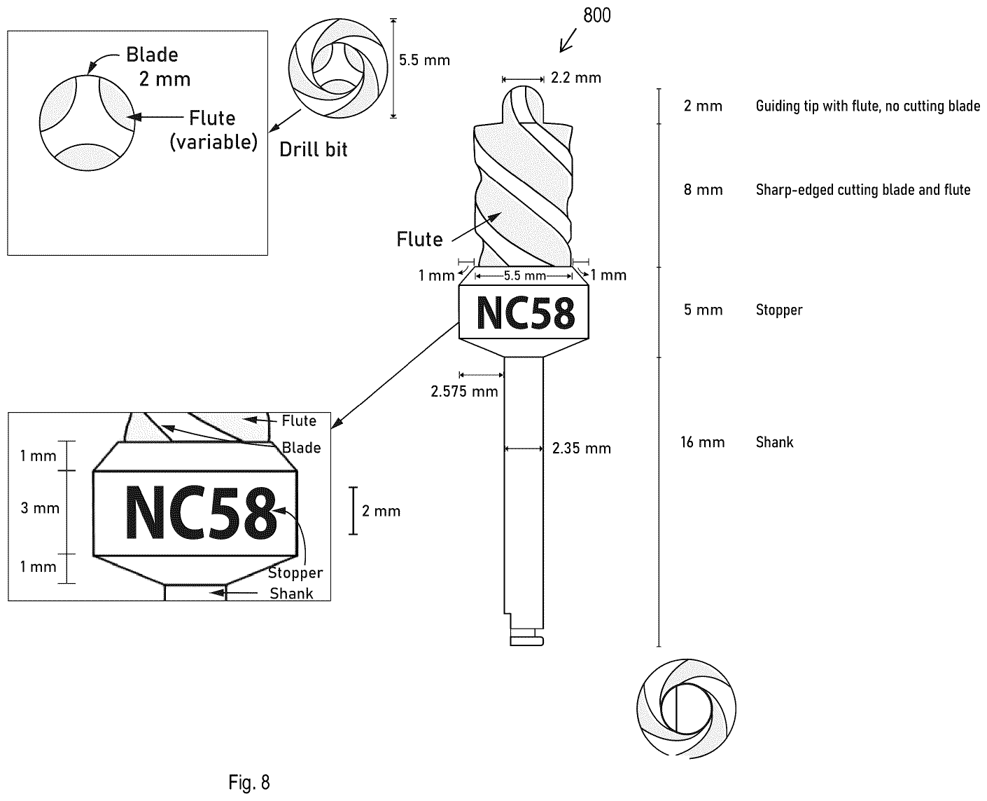

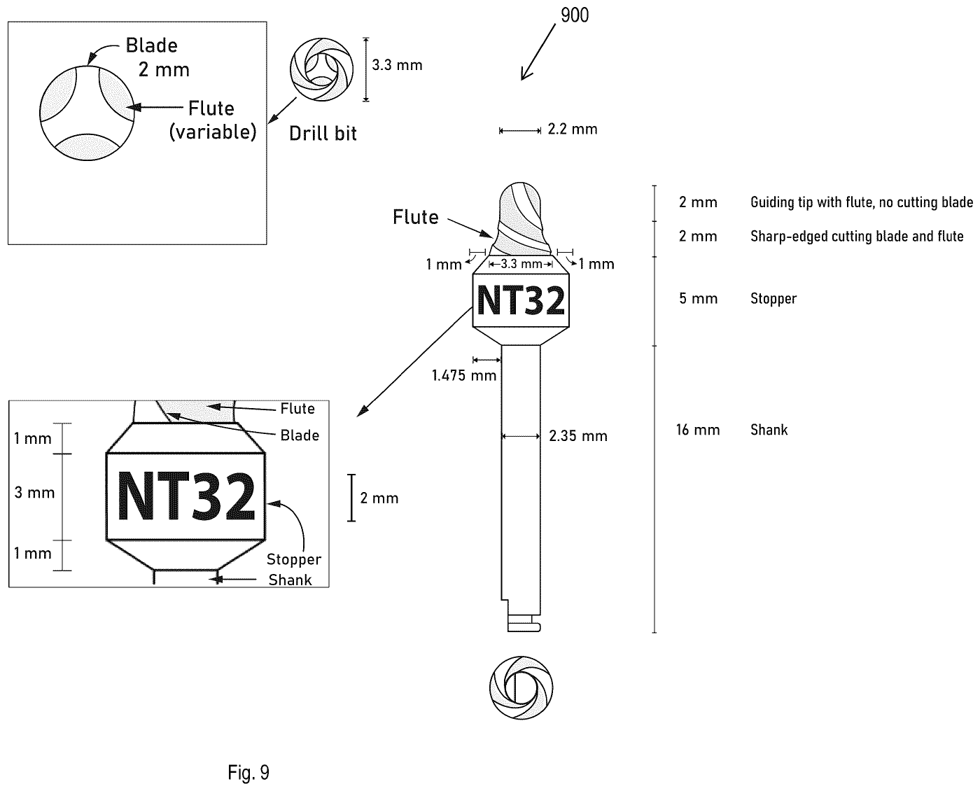

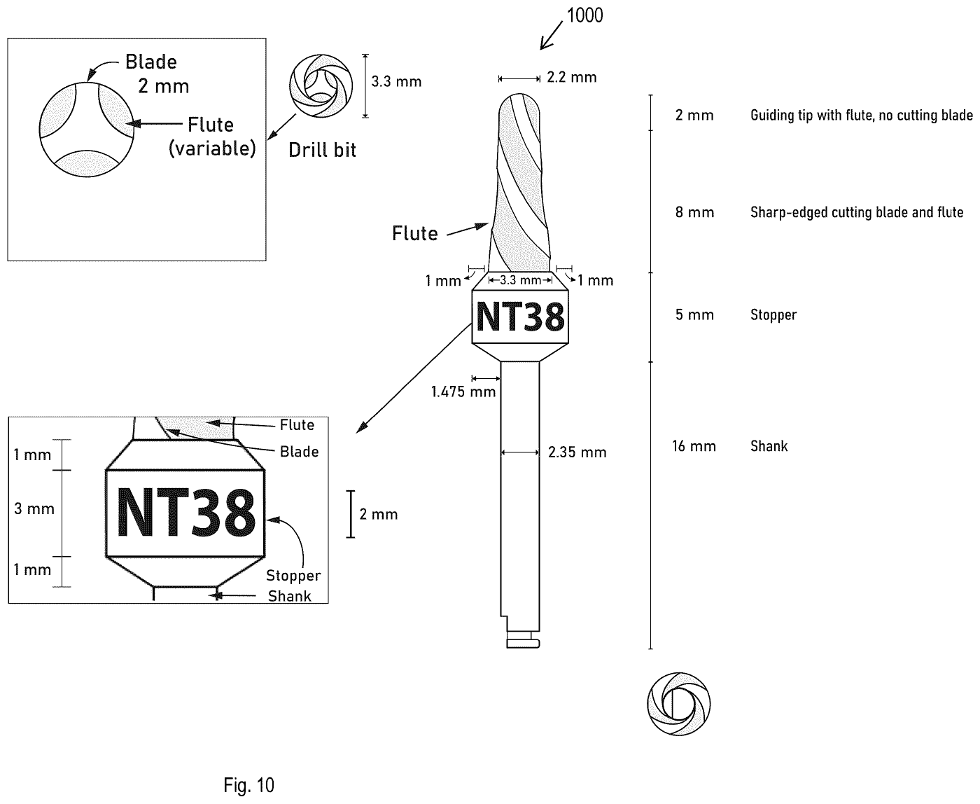

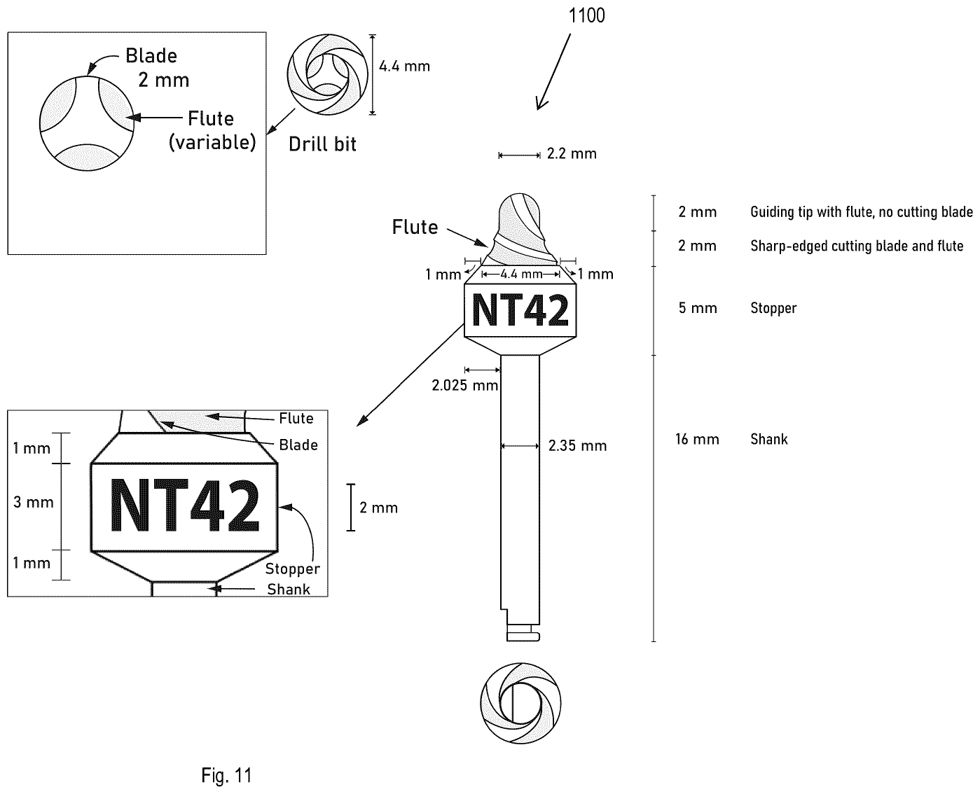

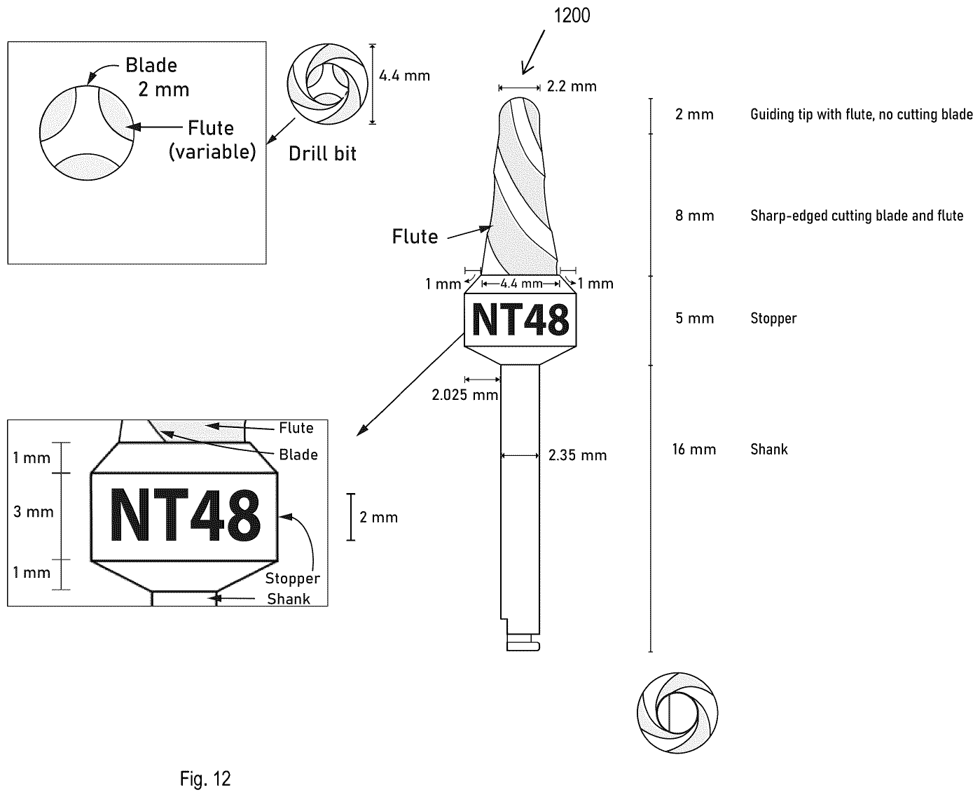

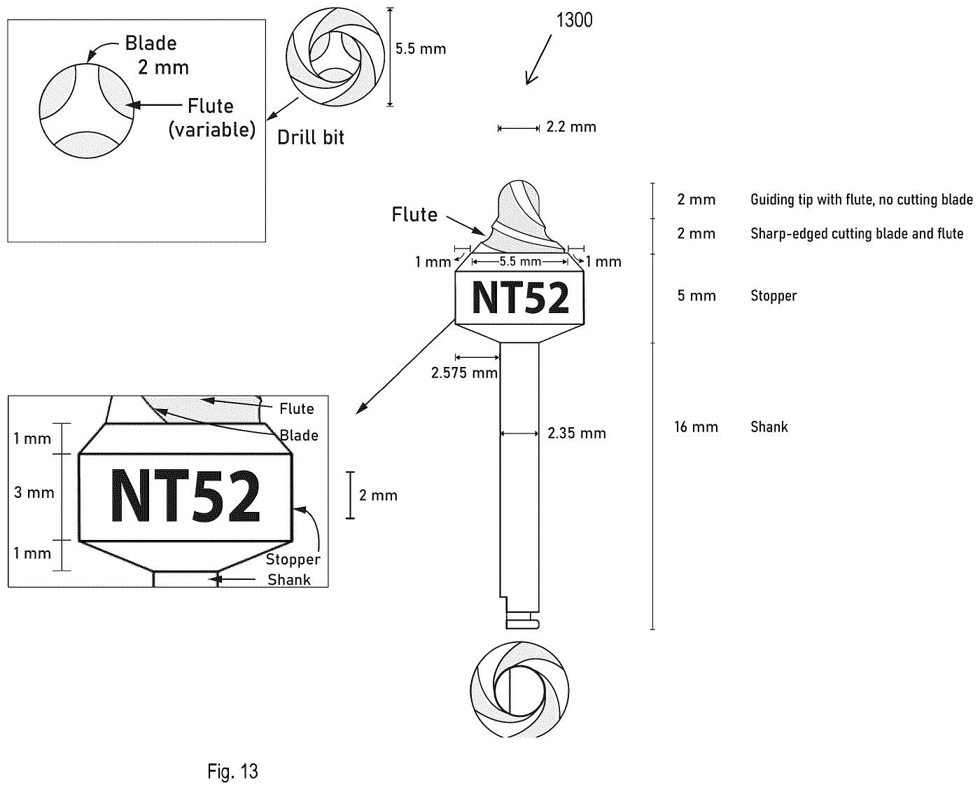

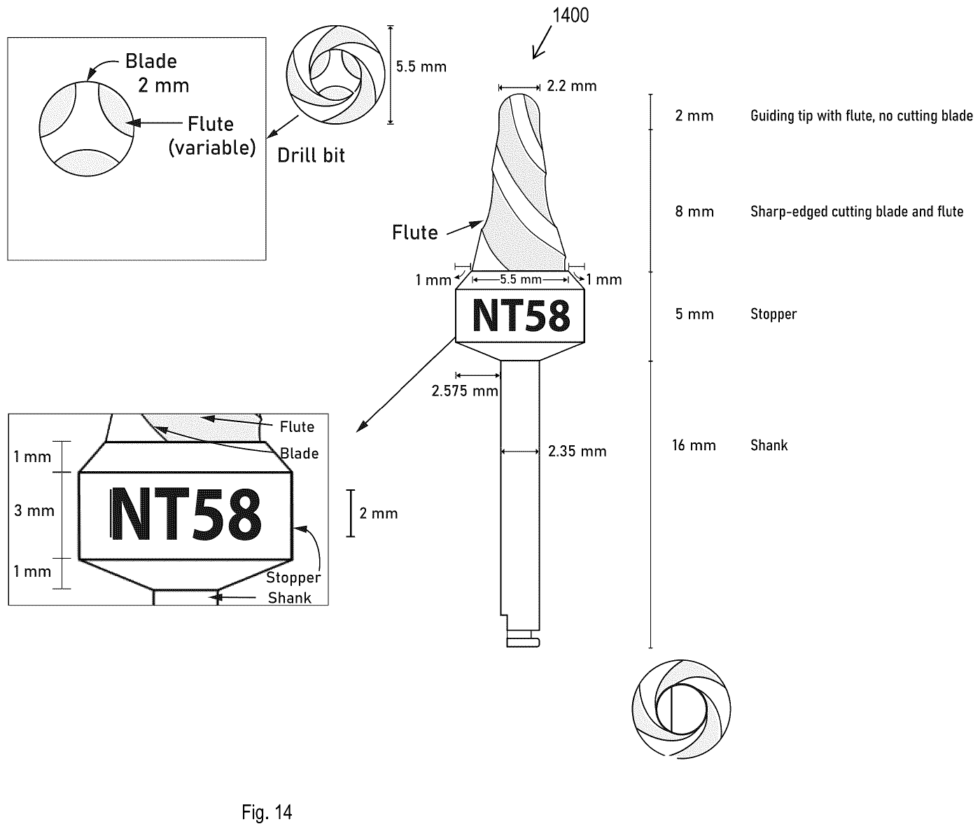

Referring to FIGS. 3-16, a system of guided implant drills is shown, with additional embodiments of guided drill bits having the upper and lower ranges of dimensions selected to correspond to standard dental implant sizes at 1 mm or 0.5 mm increments. In the embodiments, the guide portions are 2.2 mm to be compatible with standard 2.2 mm pilot drill bits. In the embodiments, straight cutting cylinder portions 300, 400, 500, 600, 700, 800 (FIGS. 3-8) vary in diameter from 3 mm to 5 mm, and vary in cutting length from 2 mm to 8 mm. In the embodiments, tapered cutting cylinder portions 900, 1000, 1100, 1200, 1300, 1400 (FIGS. 9-14) vary from a base diameter of 3.3 mm to 5.5 mm.

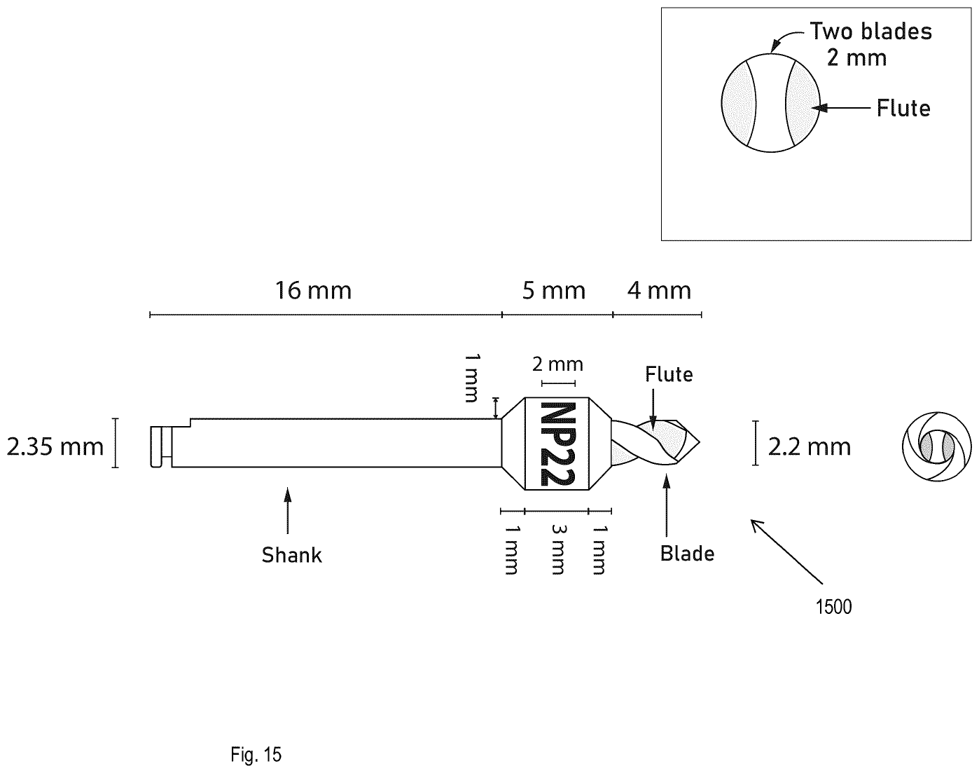

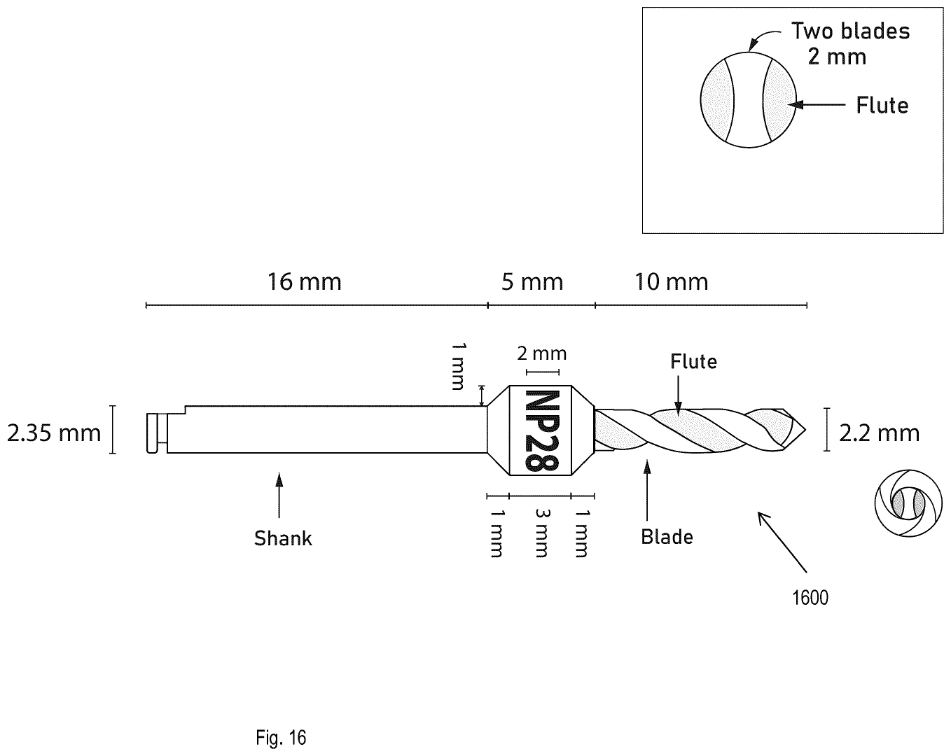

Referring to FIGS. 15-16, a range of pilot drill bits 1500, 1600 of cutting diameter 2.2 mm and depth of 4 mm (1500) to 10 mm (1600) is shown, and including a stopper portion, with additional pilot drill bits of depths at 1 mm or 0.5 mm increments.

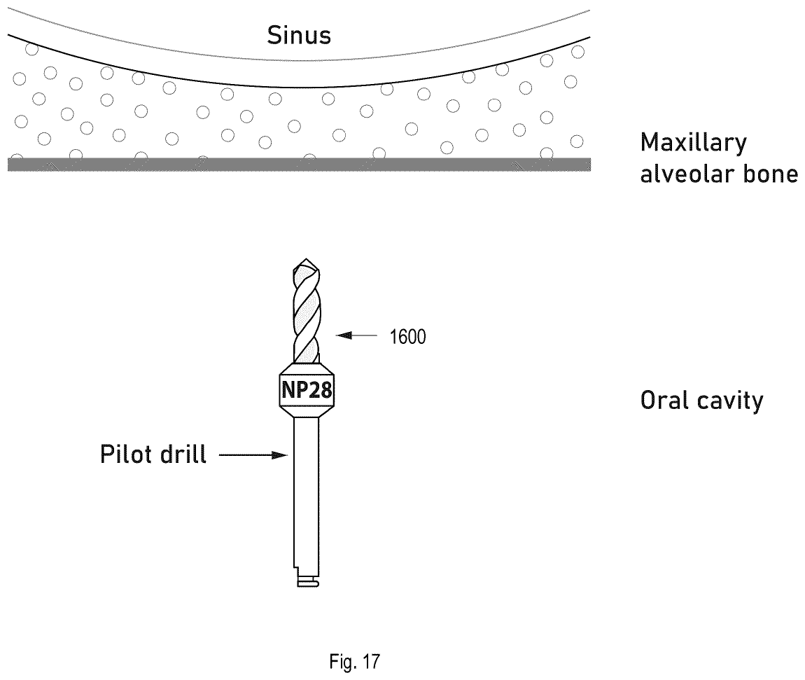

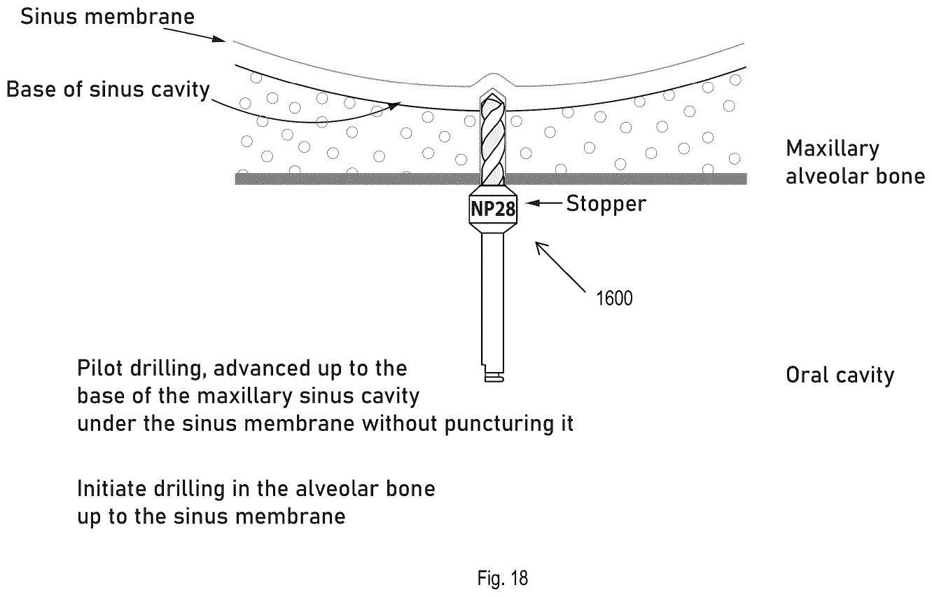

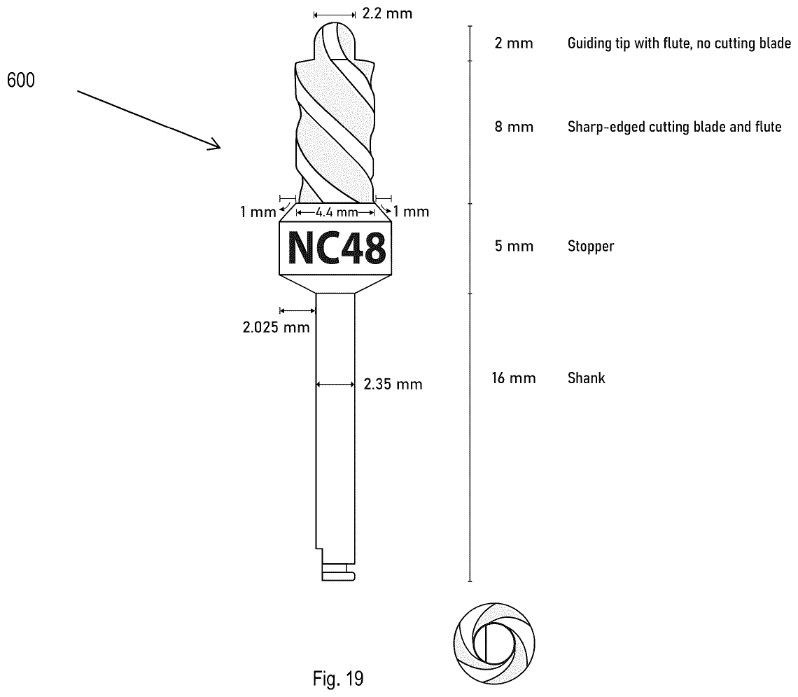

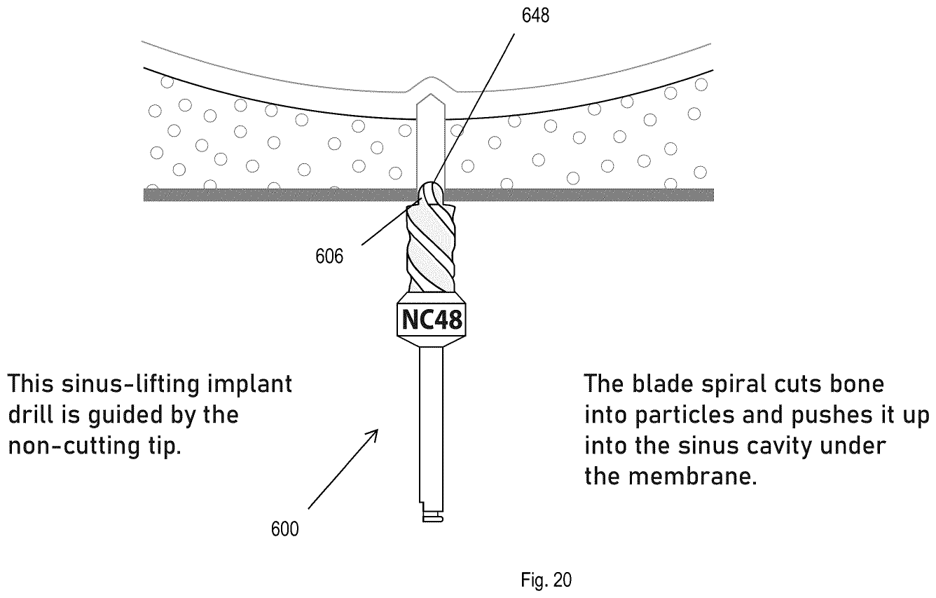

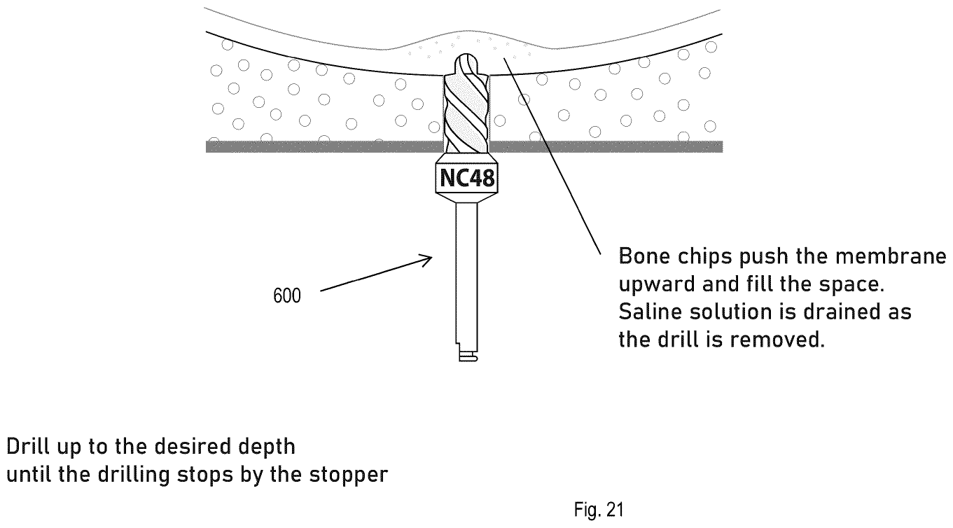

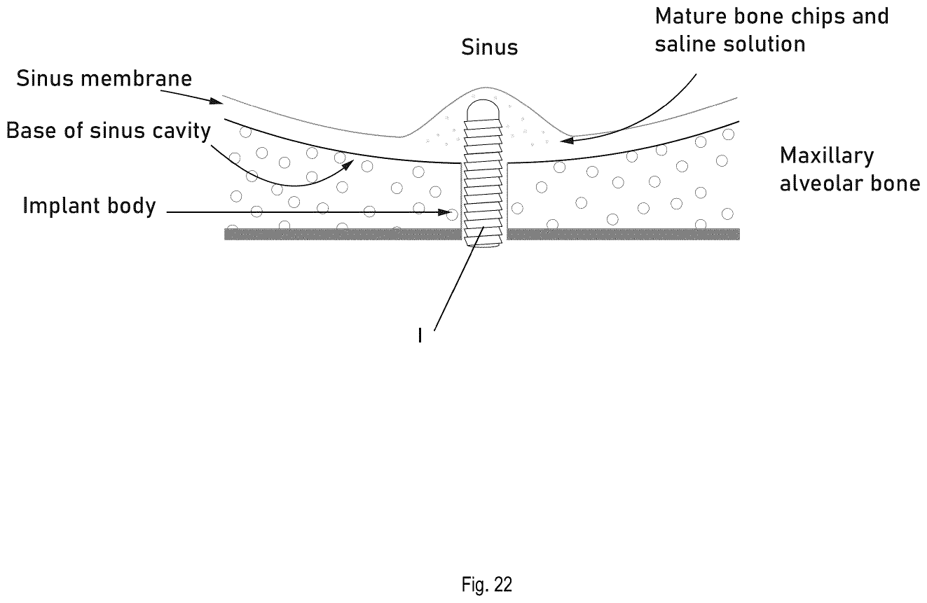

Referring to FIGS. 17-22, a method of use is shown, (the dental driver is not shown, for simplicity), including the steps of:
a. Selecting a pilot drill bit 1600 of appropriate diameter and cutting depth for the patient.
b. Creating a pilot hol at the implant location using a pilot drill 1600 of selected cutting diameter, in this case 2.2 mm, inserted until a predetermined depth is achieved thereby creating an osteotomy terminal depth. This may be done with or without resecting the gum tissue, as determined by the surgeon.
c. The surgeon selected a guided implant drill bit (300-1300), based upon the selected dental implant anchor. In the embodiment of the procedure, the surgeon selects guided implant drill bit 600 for a 4 mm diameter dental implant anchor.
d. Guided implant drill bit 600 is coupled to a selected dental driver (not shown) and used to widen the pilot hole to the desired final diameter. Guided implant drill bit guiding tip 606, having a diameter of 2.2 mm corresponding to the pilot drill bit 1600, is inserted into the pilot hole, thereby guiding the surgeon during the osteotomy. The surgeon activates the dental driver rotation in the clockwise direction and inserts guided implant drill bit 600 until the terminal end 648 of guided implant drill bit guide portion 606 reaches the osteotomy terminal depth and is then withdrawn. The noncutting surface of guiding tip 606 does not damage the maxillary sinus cavity membrane/Schneiderian membrane. The bone fragments from the osteotomy are available in situ for use as autogenous bone material to support osseointegration.
e. The osteotomy site would then be ready for the remainder of the implantation procedure for the pre-selected dental implant anchor upon executing the preceding method, inserting the selected implant anchor I into the osteotomy site and completing the surgical procedure.

Those skilled in the art will recognize that numerous modifications and changes may be made to the exemplary embodiment(s) without departing from the scope of the claimed invention. It will, of course, be understood that modifications of the invention, in its various aspects, will be apparent to those skilled in the art, some being apparent only after study, others being matters of routine mechanical, chemical and electronic design. No single feature, function or property of the exemplary embodiment(s) is essential. Other embodiments are possible, their specific designs depending upon the particular application. As such, the scope of the invention should not be limited by the particular embodiments herein described but should be defined only by the appended claims and equivalents thereof.

I claim:

1. A guided implant drill system including one or more guided implant drill bits, each guided implant drill bit having a central axis, the guided implant drill bit further comprising a shank portion, a cutting cylinder portion and a guide portion, each aligned along the central axis:
the shank portion adapted to couple to a dental drill driver, the shank extending from a shank first end to a shank second end;
the cutting cylinder portion extending from a cutting cylinder portion first end rigidly connected to the shank portion second end to a cutting cylinder portion second end along the central axis, the cutting cylinder portion including a plurality of counterclockwise helical blades and corresponding counterclockwise helical flutes defining a plurality of cutting edges, the cutting edges defining the full diameter of the cutting cylinder portion and selected to correspond to a selected dental implant anchor, the distance from the cutting cylinder first end to the cutting cylinder second end defining the cutting cylinder length;
the guide portion extending from the cutting cylinder portion second end to a terminal end and including a constant diameter along its axial length and defining a guide portion depth, the guide portion diameter corresponding to a selected pilot drill bit cutting diameter, the guide portion including a plurality of noncutting counterclockwise helical blades and corresponding counterclockwise helical flutes, the plurality of guide portion noncutting helical blades converging at the guide portion terminal end defining a noncutting surface, each of the plurality of guide portion helical flutes in fluid communication with at least one of the plurality of helical cutting cylinder flutes.

2. A guided implant drill system as in claim 1, further comprising:
wherein the cutting cylinder diameter is constant from the cutting cylinder first end to the cutting cylinder second end.

3. A guided implant drill system as in claim 2, further comprising:
the cutting cylinder portion second end further defining a flat shoulder region surrounding the guide portion.

4. A guided implant drill bit as in claim 1, further comprising:
wherein the cutting cylinder diameter tapers from the cutting cylinder first end to the cutting cylinder second end such that the cutting cylinder diameter at the cutting cylinder second end is equal to the guide portion diameter.

5. A guided implant drill system as in claim 2, further comprising:
a plurality of guided implant drill bits, the plurality of guided implant drill bit cutting cylinder diameters being in the range of 3 mm to 5 mm and the cutting cylinder lengths being in the range of 2 mm to 8 mm.

6. A guided implant drill system as in claim 4, further comprising:
a plurality of guided implant drill bits, the plurality of guided implant drill bit cutting cylinder diameters being in the range of 3 mm to 5 mm and the cutting cylinder lengths being in the range of 2 mm to 8 mm.

7. A method for performing dental implant anchor osteotomy, comprising the steps of:
a. providing a guided implant drill system as in claim 1 corresponding to a selected dental implant anchor;
b. creating a pilot hole at the osteotomy location using a selected pilot drill bit to a predetermined depth, thereby creating an osteotomy terminal depth at the osteotomy site;
c. using the selected guided implant drill bit coupled to a dental driver set for clockwise rotation, inserting the guide portion into the pilot hole and drilling into the osteotomy location until the guided implant drill bit cutting cylinder second end reaches the osteotomy terminal depth.
